(19) United States
(12) Patent Application Publication
(10) Pub. No.: US 2007/0208317 A1
(43) Pub. Date: Sep. 6, 2007

(54) Disposable pant-like absorbent undergarment that provides close to the body fit

(76) Inventors: Patsy Ann Krautkramer, Omro, WI (US); Julie Ann Paveletzke, Appleton, WI (US); Annastacia Jane Kistler, Appleton, WI (US); Wendy Lynn Hamilton, Neenah, WI (US)

Correspondence Address: KIMBERLY-CLARK WORLDWIDE, INC., 401 NORTH LAKE STREET, NEENAH, WI, 54956, US

(21) Appl. No.: 11/367,220

(22) Filed: Mar. 3, 2006

(51) Int. Cl. A61F 13/15

(52) U.S. Cl. 604/385.3; 604/385.26

(57) ABSTRACT

A disposable pant-like absorbent undergarment is disclosed formed with a one-piece chassis having front, back and crotch regions. An absorbent assembly is secured to the chassis. A pair of seams joins the front and back regions to form a unitary undergarment having a waist opening and a pair of leg openings. The undergarment also includes leg elastics, waist elastics and chassis elastics. Each of the waist elastics is spaced apart from one another by a distance y and each has a tension value. The chassis elastics extend around the front and back regions without crossing the absorbent. Each of the chassis elastics is spaced apart from one another by a distance z and each has a tension value. Furthermore, the distance z is greater than the distance y.

### BACKGROUND OF THE INVENTION

A disposable pant-like absorbent undergarment is designed for absorbing human exudate and has a waist opening and a pair of leg openings. The disposable pant-like absorbent undergarment tries to approach the appearance, size and shape of regular cloth underwear. The disposable pant-like absorbent undergarment differs from regular cloth underwear in that it is not designed to be laundered and reused two or more times. A disposable pant-like absorbent undergarment is intended to be worn by persons, including infants, toddlers or adults and is designed for single or temporary use. The disposable pant-like absorbent undergarment is meant to be disposed of after being used once instead of being cleaned and reused. The disposable pant-like absorbent undergarment is a unitary design that is meant to be pulled up around a user's torso without having to first open the undergarment in order to place it on a user's body. The stretchability of the material used to construct the disposable pant-like absorbent undergarment permits the undergarment to snugly conform to the anatomy of the user's torso. The disposable pant-like absorbent undergarment can be manufactured to be an infant diaper, a child training pant, an adult incontinent garment, a feminine menstrual pant, etc.

Today, manufacturers are being asked by consumers to design disposable pant-like absorbent undergarments that more closely resemble cloth underwear. This is especially true for adults who are just starting to wear incontinent undergarments. Essentially, all disposable pant-like absorbent undergarments utilize an absorbent system that is designed to absorb bodily fluids, such as urine, and keep the wearer dry. The absorbent is retained close to the torso of the wearer by an enclosed chassis. The chassis can be constructed as a laminate having a plurality of elastic strands sandwiched between two outer layers. The plurality of elastic strands normally extend over about a third to about a half of the surface area of the front and back regions and simulate a wide waistband. This elastic region is capable of maintaining a consistent tension to hold the undergarment in place around the wearer's torso even after the absorbent system has been saturated with a large quantity of bodily fluid. However, the lower half to about two-thirds of the surface area of the front and back regions normally do not contain elastic strands and this non-elasticized laminate material tends to move outward, away from the wearer's torso. Such action causes two problems. First, as the non-elasticized material blossoms outward, it creates a bulbous area which can be noticeable under the wearer's outer clothing. Second, this non-elasticized material can create a rustling sound when the wearer moves about, signaling to others that this person is wearing an incontinent undergarment. For adult incontinent undergarments in particular, discretion and close to the body fit are required features that are very important to the wearer.

Now a disposable pant-like absorbent undergarment for absorbing human discharge has been invented that is discreet and provides close to the body fit.

### SUMMARY OF THE INVENTION

Briefly, this invention relates to a disposable pant-like absorbent undergarment for absorbing human body fluid and solid excrement. The disposable pant-like absorbent undergarment includes a one-piece chassis having a front region with first and second side edges, a back region with first and second side edges, and a crotch region positioned therebetween. An absorbent assembly is secured to at least one of the front, back and crotch regions and includes a liquid permeable bodyside liner, a liquid-impermeable backsheet and an absorbent positioned therebetween. The absorbent assembly and the chassis are capable of being folded to enable the front region to overlap the back region. A pair of seams joins the first and second side edges of the front region to the first and second side edges of the back region, respectively, to form a unitary undergarment having a waist opening and a pair of leg openings. The undergarment also includes leg elastics, waist elastics and chassis elastics. The leg elastics are secured to the front and back regions of the chassis and at least partially surround each of the pair of leg openings. The waist elastics are secured to and extend circumferentially around the front and back regions of the chassis. The waist elastics are aligned adjacent to the waist opening. Each of the waist elastics is spaced apart from one another by a distance y and each has a tension value. The chassis elastics are secured to and extend around the front and back regions of the chassis without crossing the absorbent. The chassis elastics extend from between the waist elastics to a portion of the leg elastics, without intersecting the leg elastics. Each of the chassis elastics is spaced apart from one another by a distance z and each has a tension value. The distance z is greater than the distance y and the tension value of the chassis elastics is less than the tension value of the waist elastics.

### BRIEF DESCRIPTION OF THE DRAWINGS

FIG. 1 is a perspective view of a disposable pant-like absorbent undergarment having leg elastics, waist elastics and chassis elastics that provide close to the body fit.

FIG. 2 is a plane view of the disposable pant-like absorbent undergarment shown in FIG. 1 before the pair of seams are formed and includes a partial cut away section depicting the makeup of the absorbent assembly.

FIG. 3 is a side view of the disposable pant-like absorbent undergarment shown in FIG. 1 and taken along line 3-3.

FIG. 4 is an enlarged view of a portion of the back region of the undergarment shown in FIG. 2 depicting the spacing of the elastic strands forming the waist elastics and the chassis elastics.

FIG. 5 is a view of a person's buttock and rear torso when wearing the disposable pant-like absorbent undergarment shown in FIG. 1 depicting three different elastic spacings.

### DETAILED DESCRIPTION

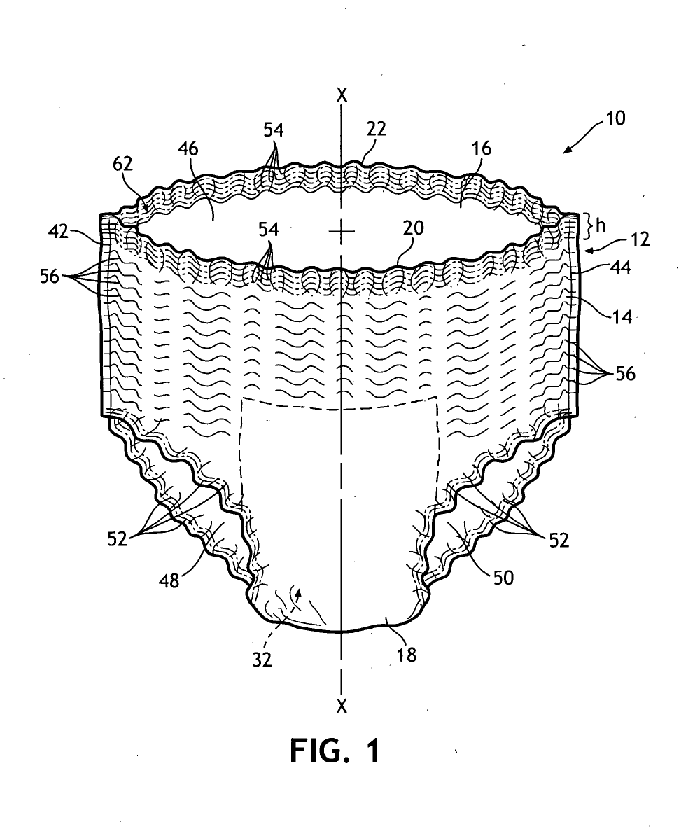

Referring to FIG. 1, a disposable pant-like absorbent undergarment 10 is depicted. The disposable pant-like absorbent undergarment 10 is intended to be worn by persons, including infants, toddlers or adults and is designed for a single or temporary use. The disposable pant-like absorbent undergarment 10 is meant to be disposed of after being used once instead of being laundered or cleaned for reuse. The disposable pant-like absorbent undergarment 10 is a unitary design that is meant to be pulled up around a user's torso without having to first open the undergarment 10 in order to place it on a user's body. In FIG. 1, the disposable pant-like absorbent undergarment 10 is shown as it would appear after it has been removed from its package but before it is pulled up around a user's torso.

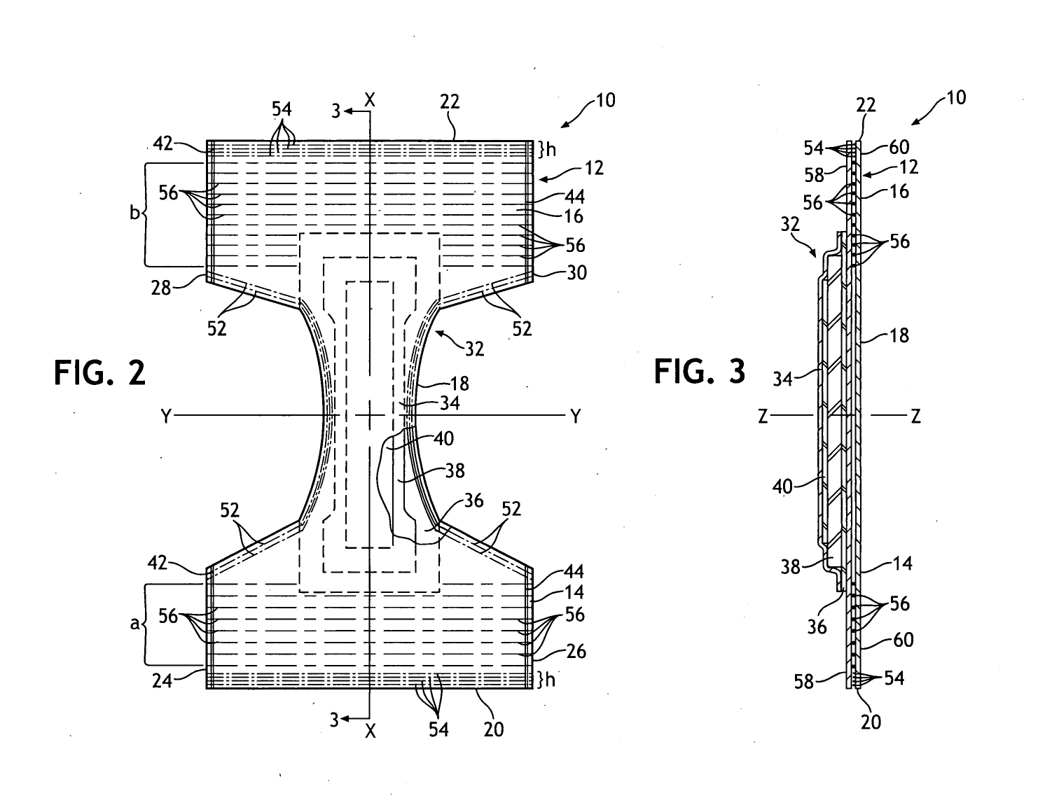

Referring to FIGS. 2 and 3, the disposable pant-like absorbent undergarment 10 is depicted in an open configuration solely for the purpose of better showing the various components. The open undergarment 10 has a longitudinal central axis X-X, a transverse central axis Y-Y and a vertical central axis Z-Z. The disposable pant-like absorbent undergarment 10 includes a one-piece chassis 12 which can be liquid permeable or liquid-impermeable. Desirably, the chassis 12 is liquid permeable such that a liquid can easily pass therethrough. The chassis 12 can be formed from various materials known to those skilled in the art. Desirably, the chassis 12 is a laminate having at least two layers. The chassis 12 includes a front region 14, a back region 16 and a crotch region 18 formed therebetween. The chassis 12 also has a first end 20 and a second end 22. The ends 20 and 22 are spaced apart from one another. In FIG. 2, the first end 20 is a distal end of the front region 14 while the second end 22 is a distal end of the back region 16. The front region 14 further has a first side edge 24 and a second side edge 26. Likewise, the back region 16 has a first side edge 28 and a second side edge 30.

Still referring to FIGS. 2 and 3, the disposable pant-like absorbent undergarment 10 also includes an absorbent assembly 32 secured to at least one of the regions 14, 16 and 18 of the chassis 12. Desirably, the absorbent assembly 32 is secured to at least two of the regions 14, 16 and 18 of the chassis 12. Even more desirably, the absorbent assembly 32 is secured to all three regions 14, 16 and 18 of the chassis 12. The absorbent assembly 32 can be secured to one or more regions 14, 16 and 18 of the chassis 12 by an adhesive or by other means known to those skilled in the art. The absorbent assembly 32 includes a liquid permeable bodyside liner 34, a liquid-impermeable backsheet or baffle 36 and an absorbent 38 positioned therebetween. The bodyside liner 34 can be formed from a material that will allow liquids, especially urine, to pass quickly therethrough. The bodyside liner 34 can be formed from natural or synthetic materials that are either woven or non-woven. Spunbond is a nonwoven material that functions well as the bodyside liner 34. Spunbond is manufactured and sold by Kimberly-Clark Corporation, having an office at 401 North Lake Street, Neenah, Wis. 54956.

The backsheet 36 can be formed from any material that will limit or prevent the passage of a liquid therethrough. Desirably, the liquid-impermeable backsheet 36 should be formed from a material that will prevent liquids, especially urine, from passing therethrough. Some materials that function well as a backsheet 36 include synthetic materials, such as plastic and thermoplastic materials, especially films; polyolefins, such as polyethylene and polypropylene; and other materials known to those skilled in the art. The backsheet 36 can be relatively thin, having a thickness of only a few millimeters, and should have a soft hand and be flexible. A film material that is quiet when deformed works best.

The absorbent 38 can be formed from natural or synthetic materials. The absorbent 38 can be made from cellulosic fibers, wood pulp, textile fibers or from other absorbent materials known to those skilled in the art. Superabsorbents, commonly in solid form and in the shape of small particles, granules, flakes, etc., can be mixed, combined, attached, printed or otherwise added to the absorbent material to increase the absorbent capacity of the absorbent 38. A surge layer 40 can also be optionally used, which is normally positioned between the bodyside liner 34 and the absorbent 38. The surge layer 40 functions to rapidly acquire and temporarily retain body fluid, such as urine, before it can be absorbed into the absorbent 38. Desirably, the surge layer 40 is capable of wicking body fluid lengthwise and/or widthwise across its surface as well as directing the body fluid downward in a z-direction, into the absorbent 38.

It should be noted that the absorbent assembly 32 can be constructed with two or more layers of absorbent 38, if desired. Any or all of the layers can contain a superabsorbent material. Likewise, the absorbent assembly 32 can also be constructed without the bodyside liner 34, without the backsheet 36 or without either layer 34 and 36, if one so desires.

The crotch region 18 of the chassis 12 and the absorbent assembly 32 are capable of being folded to enable the front region 14 to overlap the back region 16. When the crotch region 18 is folded, the first and second ends, 20 and 22 respectively, of the chassis 12 will be approximately even. In FIG. 2, the crotch region 18 and the absorbent assembly 32 can be folded along the transverse centerline Y-Y to yield the profile shown in FIG. 1 wherein the front region 14 overlies the back region 16.

Referring again to FIGS. 1 and 2, the disposable pant-like absorbent undergarment 10 further includes a pair of seams 42 and 44 joining the first and second side edges, 24 and 26 respectively, of the front region 14 to the first and second side edges, 28 and 30 respectively, of the back region 16. Once the pair of seams 42 and 44 is formed, the disposable pant-like absorbent undergarment 10 acquires a unitary design having a waist opening 46 and a pair of leg openings 48 and 50. By a “unitary design” it is meant that the undergarment 10 functions similar to regular cloth underwear as has to be pulled up along the wearer's leg and torso. Once the pair of seams 42 and 44 are broken, the disposable pant-like absorbent undergarment 10 can not easily be reused and must be discarded.

Turning again to FIGS. 2 and 3, the disposable pant-like absorbent undergarment 10 further includes leg elastics 52, waist elastics 54 and chassis elastics 56. The leg elastics 52 can include from between 2 to 5 individual elastic strands that are spaced apart from one another by a distance x. The distance x can range from between about 1 millimeter (mm) to 5 mm. Desirably, the distance x will be from between about 2 mm to about 4 mm. Each of the leg elastics 52 also has a tension value. The exact tension value of each of the leg elastics 52 can vary depending upon the particular undergarment being produced. Tension can be controlled by varying a number of items including but not limited to: the spacing between each elastic strand, the material from which the leg elastics 52 are formed, the number of elastic strands present, the stretch imparted into each of the elastic strands before they are secured to the front and back regions, 14 and 16 respectively, the decitex (weight in grams per 10,000 meters of a strand) of each of the elastic strands, the cross-sectional area of each of the elastic strands, the means employed for securing each of the elastic strands to the chassis 12, etc. One skilled in the art will know how these and other variables affect the tension value of the leg elastics 52.

The leg elastics 52 are positioned adjacent to each of the leg openings 48 and 50 and are secured to the front and back regions, 14 and 16 respectively, of the chassis 12. The individual elastic strands which form the leg elastics 52 can be formed from CREORA SPANDEX or LYCRA or from any other elastomeric material known to those skilled in the art. CREORA SPANDEX is a registered trademark of Hyosung Corporation, having an office at 450, Gongdeok-dong, Mapo-gu, Seoul, Korea (121-720). LYCRA is a registered trademark of Invista, having an office at 4123 East 37^{th} St. North, Wichita, Kansas 67220. The individual elastic strands should be aligned approximately parallel to one another or they can be aligned at a slight angle or be skewed relative to one another. However, the elastic strands of the leg elastics 52 should not overlap, cross or intersect one another. The elastic strands can vary in shape, size, configuration and/or length. The diameter and/or cross-sectional configuration of the elastic strands, the decitex of the elastic strands, and the tension imparted into the elastic strands can all be varied to suit one's particular needs. The individual elastic strands can have a round, semi-circular, square, rectangular, oval or some other geometrical configuration. The various ways of positioning, orienting and/or adhering the individual elastic strands to the front and back regions, 14 and 16 respectively, of the chassis 12 are well known to those skilled in the art.

The leg elastics 52 are positioned from between about 0.1 to about 0.5 inches (about 2.5 mm to about 13 mm) from a terminal edge of each of the leg openings 48 and 50. The leg elastics 52 at least partially surround each of the leg openings 48 and 50. Desirably, the leg elastics 52 will surround at least about 75% of the periphery of each of the leg openings 48 and 50. More desirably, the leg elastics 52 will surround at least about 90% of the periphery of each of the leg openings 48 and 50. Even more desirably, the leg elastics 52 will completely surround each of the leg openings 48 and 50.

Turning to FIGS. 2 and 3, as was mentioned above, the chassis 12 can be formed as a laminate having at least two layers, including an inner layer 58 and an outer layer 60. The inner and outer layers, 58 and 60 respectively, can be constructed or formed from a woven or a nonwoven material, a natural or synthetic material, an elastic film, a thermoplastic film, or from any other material known to those skilled in the art. Desirably, the chassis 12 should be constructed from a material having at least a minimum amount of stretch and/or retraction capabilities. A nonwoven material from which the chassis 12 can be constructed is spunbond. In FIG. 2, the leg elastics 52 are shown sandwiched between the inner and outer layers, 58 and 60 respectively, of the front and back regions, 14 and 16, of the chassis 12.

Referring again to FIGS. 1-3, the undergarment 10 also includes waist elastics 54 that are secured to the front and back regions, 14 and 16 respectively, of the chassis 12. The waist elastics 54 can include from between 2 to 10 individual elastic strands that are aligned approximately parallel to one another. Desirably, the waist elastics 54 should include from between 2 to 6 individual elastic strands. More desirably, the waist elastics 54 include at least 5 individual elastic strands. Even more desirably, the waist elastics 54 include at least 6 individual elastic strands.

Each of the waist elastics 54 also has a tension value. The exact tension value of each of the leg elastics 52 can vary depending upon the particular undergarment being produced. The tension value of the individual elastic strands forming the waist elastics 54 can be less than, equal to, or be greater than the tension value of the individual elastic strands forming the leg elastics 52. Desirably, the tension value of the individual elastic strands forming the waist elastics 54 will be less than or equal to the tension value of the individual elastic strands forming the leg elastics 52. The tension value of each of the individual elastic strands forming the waist elastics 54 can be selected by varying a number of items: the spacing between each elastic strand, the material from which the waist elastics 54 are formed, the number of elastic strands present, the stretch imparted into each of the elastic strands before they are secured to the front and back regions, 14 and 16 respectively, the decitex of each of the elastic strands, the cross-sectional area of each of the elastic strands, the means employed for securing each of the elastic strands to the chassis 12, etc. One skilled in the art will know how these and other variables affect the tension value of the waist elastics 54.

It should be noted that the waist elastics 54 used in an adult incontinent undergarment will most likely have a higher tension value than the waist elastics 54 utilized in an infant diaper or in a child training pants. Diapers and training pants are designed to be placed on an infant and toddler, respectively, which have relatively small body dimensions and, therefore, the tension value can be reduced since a smaller amount of tension is needed to retain the garment in place around the wearer's torso.

The individual elastic strands forming the waist elastics 54 can be formed from CREORA SPANDEX, as stated above in relation to the leg elastics 52, or they can be formed from any other elastomeric material known to those skilled in the art. The waist elastics 54 extend from the first side edge 24 to the second side edge 26 of the front region 14 and from the first side edge 28 to the second side edge 30 of the back region 16. Desirably, the waist elastics 54 are continuous elastic strands. The waist elastics 54 extend completely across the pair of seams 42 and 44 once the front region 14 is overlaid and bonded to the back region 16. As depicted in FIG. 1, in the unitary undergarment 10, the waist elastics 54 extend circumferentially around the front and back regions, 14 and 16 respectively, of the chassis 12. Another way of stating this is to say that the waist elastics 54 extend 360 degrees around the periphery of the waist opening 46. Furthermore, the waist elastics 54 can extend into and across the material which forms the pair of seams 42 and 44, if desired.

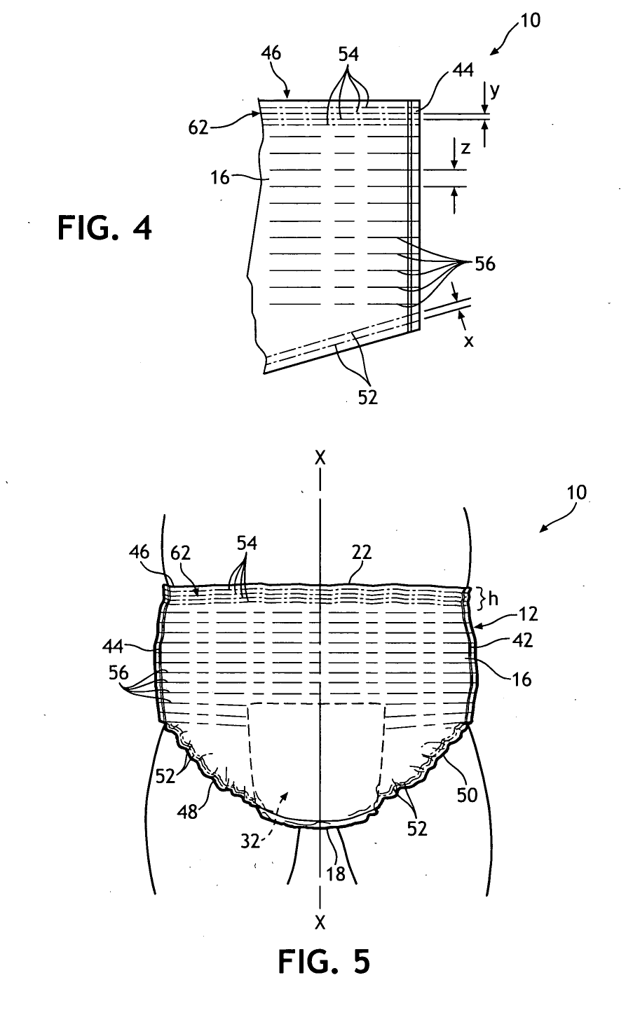

Referring now to FIG. 4, the individual elastic strands forming the waist elastics 54 are spaced apart from one another by a distance y. The distance y can vary but should range from between about 1 mm to about 7.5 mm. Desirably, the individual elastic strands forming the waist elastics 54 are spaced apart from one another by a distance y that ranges from between about 3 mm to about 7 mm. More desirably, the individual elastic strands forming the waist elastics 54 are spaced apart from one another by a distance y that is less than about 7 mm. Even more desirably, the distance y should be about 6.4 mm.

In FIG. 1, the waist elastics 54 are depicted as consisting of four individual elastic strands aligned adjacent to the waist opening 46 and approximately parallel to one another. Although the individual elastic strands constituting the waist elastics 54 do not have to be exactly parallel to one another, they should not intersect or overlap. The reason for this is that separate and distinct elastic strands create a more aesthetically pleasing appearance. The entirety of the waist elastics 54 creates a waist band 62 around the periphery of the waist opening 46. The waist band 62 can have a height h ranging from between about 0.5 inches to about 2 inches (about 13 mm to about 51 mm). Desirably, the waist band 62 will have a height h of from between about 0.6 inches to about 1.5 inches (about 15 mm to about 38 mm). Even more desirably, the waist band 62 will have a height h of from between about 0.6 inches to about 1.5 inches (about 15 mm to about 38 mm). Most desirably, the waist band 62 will have a height h of about 1 inch (about 25 mm).

Returning again to FIGS. 1-3, the undergarment 10 further includes chassis elastics 56 that are secured to the front and back regions, 14 and 16 respectively, of the chassis 12. The chassis elastics 56 should contain at least 12 individual elastic strands that are aligned approximately parallel to one another. Desirably, the chassis elastics 56 should contain at least 18 individual elastic strands. More desirably, the chassis elastics 56 will include from between about 18 to about 30 individual elastic strands. More desirably, the chassis elastics 56 will include from between about 18 to about 24 individual elastic strands. The chassis elastics 56 are aligned approximately perpendicular to the longitudinal central axis X-X and extend from the waist elastics 54 to a portion of the leg elastics 52. The chassis elastics 56 do not cross or intersect the leg elastics 52. By doing so could compromise the function of the leg elastics 52. The chassis elastics 56 form a continuous elastic area between the waist elastics 54 and at least a portion of the leg elastics 52. Desirably, there are no gaps or segments greater than about 0.5 inches (about 13 mm) in width that do not contain individual elastic strands.

Referring again to FIG. 2, one will notice that the individual elastic strands forming the chassis elastics 56 in the front region 14 can extend over a distance “a”. Likewise, the individual elastic strands forming the chassis elastics 56 in the back region 16 can extend over a distance “b”. Desirably, the distance a will be approximately equal the distance b. Alternatively, the distance a will be less than or be greater than the distance b. In either case, it is beneficial when the elastic strands in both the front and back regions, 14 and 16 respectively, that are located farthest from the respective end 20 or 22, align with one another. This feature creates a very aesthetic appearance in the finished disposable pant-like absorbent undergarment 10.

It should be noted that the chassis elastics 56 cover at least about 50% of the total surface area of each of said front and back regions, 14 and 16 respectively. The total surface area of each of the front and back regions, 14 and 16 respectively, extend from the respective end 20 or 22 to the crotch region 18. The crotch region 18 terminates approximately at the inner terminal ends of the leg elastics 52. Desirably, the chassis elastics 56 cover at least about 60% of the total surface area of each of said front and back regions, 14 and 16 respectively. More desirably, the chassis elastics 56 cover at least about 70% of the total surface area of each of said front and back regions, 14 and 16 respectively. Even more desirably, the chassis elastics 56 cover at least about 80% of the total surface area of each of said front and back regions, 14 and 16 respectively.

Each of the chassis elastics 56 also has a tension value. The exact tension value of each of the chassis elastics 56 can vary depending upon the particular undergarment being produced. However, the tension value of the individual elastic strands forming the chassis elastics 56 should be less than the tension values of the elastic strands forming the waist elastics 54. In addition, the tension value of the individual elastic strands forming the chassis elastics 56 should be less than the tension values of the elastic strands forming the leg elastics 52.

The elastic strands forming the chassis elastics 56 can be formed from CREORA SPANDEX, as stated above in relation to the leg elastics 52 and waist elastics 54, or they can be formed from any other elastomeric material known to those skilled in the art. The chassis elastics 56 also extend from the first side edge 24 to the second side edge 26 of the front region 14 and from the first side edge 28 to the second side edge 30 of the back region 16. Desirably, the chassis elastics 56 are continuous elastic strands. Desirably, at least some of the chassis elastics 56 extend completely across the pair of seams 42 and 44 once the front region 14 is overlaid and bonded to the back region 16. Alternatively, the chassis elastics 54 can stop short of the material forming the pair of seams 42 and 44. As depicted in FIGS. 1 and 2, in the unitary undergarment 10, the chassis elastics 56 extend around the front and back regions, 14 and 16 respectively, of the chassis 12 without crossing the absorbent assembly 32. This feature is important because the absorbent assembly 32 should lie flat and close to the torso of the wearer so as to receive any body fluids discharged by the wearer. If the chassis live elastics 56 were allowed to cross over the absorbent assembly 32, they could create gathers, wrinkles, rugosities or ripples in the absorbent assembly 32 which could mitigate the function of the absorbent assembly 32.

Referring again to FIG. 4, the individual elastic strands forming the chassis elastics 56 are spaced apart from one another by a distance z. The distance z can vary but should range from between about 4 mm to about 12 mm. Desirably, the distance z should range from between about 6 mm to about 10 mm. More desirably, the distance z should be more than 7 mm. Even more desirably, the distance z should be about 8.3 mm. The distance z is greater than the distance x, which separates the individual elastic strands of the leg elastics 52. Furthermore, the distance z is greater than the distance y, which separates the individual elastic strands of the waist elastics 54.

Since the chassis elastics 56 cover a greater surface area of the front and back regions, 14 and 16 respectively, of the chassis 12, the number of individual elastic strands forming the chassis elastics 56 greatly exceeds the number of elastic strands forming the waist elastics 54. Desirably, there are at least three times as many individual elastic strands in the chassis elastics 56 as there are in the waist elastics 54. More desirably, there are at least six times as many individual elastic strands in the chassis elastics 56 as there are in the waist elastics 54. Likewise, the number of individual elastic strands forming the chassis elastics 56 greatly exceeds the number of elastic strands forming the leg elastics 52. Desirably, there are at least six times as many individual elastic strands in the chassis elastics 56 as there are in the leg elastics 52.

Lastly, referring now to FIG. 5, the disposable pant-like absorbent undergarment 10 is shown positioned around the buttocks and torso of a wearer. One will quickly recognize that the combination of the leg elastics 52, the waist elastic 54 and the chassis elastics 56 provides a close to the body fit. There is very little material that is not elasticized that can blossom outward away from the wearer's body. This makes the disposable pant-like absorbent undergarment 10 more discreet since it is harder to notice it under outer clothing. In addition, the closer to the body fit also increases the ability of the undergarment 10 to function better in preventing fluid leakage. Since the chassis elastics 56 extends vertically, as measured along the longitudinal central axis X-X, from the waist elastics 54 down to at least a portion of the leg elastics 52, they are able to hold the absorbent assembly 32 snugly against the body of the wearer. Experimental tests have proven that, when the absorbent assembly 32 is retained up against the body of the wearer, leakage can be minimized.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A disposable pant-like absorbent undergarment comprising:
a) a one-piece chassis having a front region, a back region and a crotch region positioned therebetween;
b) an absorbent assembly secured to at least one of said front, back and crotch regions;
c) a pair of seams joining said front region to said back region to form a unitary undergarment having a waist opening and a pair of leg openings;
d) leg elastics secured to said front and back regions, said leg elastics at least partially surrounding each of said pair of leg openings;
e) waist elastics secured to and extending circumferentially around said front and back regions, said waist elastics being aligned adjacent to said waist opening, and each of said waist elastics being spaced apart from one another by a distance y and each having a tension value; and
f) chassis elastics secured to and extending around said front and back regions without crossing said absorbent assembly, said chassis elastics extending from between said waist elastics to a portion of said leg elastics, each of said chassis elastics being spaced apart from one another by a distance z and each having a tension value, said distance z being greater than said distance y, and said tension value of said chassis elastics being less than said tension value of said waist elastics.

2. The disposable pant-like absorbent undergarment of claim 1 wherein said leg elastics are spaced apart from one another by a distance x which is less than said distance y, and each of said leg elastics has a tension value which is equal to said tension value of said waist elastics.

3. The disposable pant-like absorbent undergarment of claim 1 wherein said chassis elastics contain at least three times as many elastic strands as said waist elastics.

4. The disposable pant-like absorbent undergarment of claim 1 wherein said chassis elastics contain at least six times as many elastic strands as said leg elastics.

5. The disposable pant-like absorbent undergarment of claim 1 wherein said chassis elastics extend across said pair of seams.

6. The disposable pant-like absorbent undergarment of claim 1 wherein said waist elastics include from between 2 to 10 elastic strands aligned approximately parallel to one another.

7. The disposable pant-like absorbent undergarment of claim 6 wherein said waist elastics include from between 2 to 6 elastic strands spaced less than 7 millimeters apart.

8. The disposable pant-like absorbent undergarment of claim 1 wherein said chassis elastics include at least 18 elastic strands aligned approximately parallel to one another.

9. The disposable pant-like absorbent undergarment of claim 8 wherein said chassis elastics include from 18 to 24 elastic strands spaced more than 7 millimeters apart.

10. A disposable pant-like absorbent undergarment comprising:
a) a one-piece chassis having a front region, a back region and a crotch region positioned therebetween;
b) an absorbent assembly secured to at least one of said front, back and crotch regions and including a liquid permeable bodyside liner, a liquid-impermeable backsheet and an absorbent positioned therebetween;
c) a pair of seams joining said front region to said back region to form a unitary undergarment having a waist opening and a pair of leg openings;
d) leg elastics secured to said front and back regions, said leg elastics at least partially surrounding each of said pair of leg openings;
e) waist elastics secured to and extending circumferentially around said front and back regions, said waist elastics being aligned adjacent to said waist opening, and each of said waist elastics being spaced apart from one another by a distance y and each having a tension value; and
f) chassis elastics secured to and extending around said front and back regions without crossing said absorbent assembly, said chassis elastics including at least 12 elastic strands extending from said waist elastics to a portion of said leg elastics without intersecting said leg elastics, each of said chassis elastics being spaced apart from one another by a distance z and each having a tension value, said distance z being greater than said distance y, and said tension value of said chassis elastics being less than said tension value of said waist elastics.

11. The disposable pant-like absorbent undergarment of claim 10 wherein said leg elastics are spaced apart from one another by a distance x which is less than said distance z.

12. The disposable pant-like absorbent undergarment of claim 10 wherein each of said chassis elastics has a tension value which is less than said tension value of said leg elastics.

13. The disposable pant-like absorbent undergarment of claim 10 wherein said front region has a total surface area and said chassis elastics cover at least about 50% of said total surface area of said front region.

14. The disposable pant-like absorbent undergarment of claim 10 wherein chassis elastics contain at least six times as many elastic strands as said leg elastics and at least three times as many elastic strands as said waist elastics.

15. The disposable pant-like absorbent undergarment of claim 10 wherein said waist elastics have a tension value equal to said tension value of said leg elastics and greater than said tension value of said chassis elastics.

16. A disposable pant-like absorbent undergarment comprising:
a) a one-piece liquid permeable chassis having a front region, a back region and a crotch region positioned therebetween;
b) an absorbent assembly secured to at least one of said front, back and crotch regions and including a liquid permeable bodyside liner, a liquid-impermeable backsheet and an absorbent positioned therebetween, said absorbent assembly and chassis capable of being folded to enable said front region to overlap said back region;
c) a pair of seams joining said front region to said back region to form a unitary undergarment having a waist opening and a pair of leg openings;
d) leg elastics secured to said front and back regions, said leg elastics at least partially surrounding each of said pair of leg openings;
e) waist elastics secured to and extending circumferentially around said front and back regions, said waist elastics being aligned adjacent to said waist opening and approximately parallel to one another, and each of said waist elastics being spaced apart from one another by a distance y and each having a tension value; and
f) chassis elastics secured to and extending around said front and back regions without crossing said absorbent assembly, said chassis elastics including at least 18 elastic members extending from said waist elastics to a portion of said leg elastics without intersecting said leg elastics, each of said chassis elastics being spaced apart from one another by a distance z and each having a tension value, said distance z being greater than said distance y, and said tension value of said chassis elastics being less than said tension value of said waist elastics.

17. The disposable pant-like absorbent undergarment of claim 16 wherein said leg elastics are spaced apart from one another by a distance x which is less than said distance y.

18. The disposable pant-like absorbent undergarment of claim 16 wherein said front and back regions each have a total surface area and said chassis elastics cover at least about 50% of said total surface area of each region.

19. The disposable pant-like absorbent undergarment of claim 18 wherein said chassis elastics cover at least about 70% of said total surface area of said front region.

20. The disposable pant-like absorbent undergarment of claim 18 wherein said chassis elastics cover at least about 70% of said total surface area of said back region.
